(19) United States
(12) Patent Application Publication
(10) Pub. No.: US 2025/0325400 A1
(43) Pub. Date: Oct. 23, 2025

(54) HOT/COLD BREAST PACK

(71) Applicant: FRIDABABY, LLC, Miami, FL (US)

(72) Inventors: Chelsea HIRSCHHORN, Miami, FL (US); Janette VILLALOBOS, Miami, FL (US); Dana MCPHERSON, Miami, FL (US); Alaina DEKKER, San Francisco, CA (US)

(73) Assignee: FRIDABABY, LLC, Miami, FL (US)

(21) Appl. No.: 18/638,281

(22) Filed: Apr. 17, 2024

(51) Int. Cl. A61F 7/02; A61F 7/00

(52) U.S. Cl. CPC A61F 7/02; A61F 2007/0021; A61F 2007/0219; A61F 2007/0222; A61F 2007/0223; A61F 2007/0257; A61F 2007/0277

(57) ABSTRACT

A hot/cold breast pack has a front sheet exhibiting a first thermal conductance and a back sheet exhibiting a second thermal conductance different from the first thermal conductance. The front and back sheets are bonded together at their periphery to enclose a gel. The user selects one of the front sheet and the back sheet for application to the breast to deliver heat or cold therapy.

### BACKGROUND OF THE INVENTION

It is known to apply heat to the human female breast prior to and during expression to encourage “letdown” from the milk glands in the breast. Further, it is known to apply cold to the breast to discourage “engorgement” at other times. Optimal cooling for mitigating engorgement is in the range of 10 to 18 C for about 15 to 20 minutes. Arora, S., et al., IJCM 2008, 33(3), 160. Optimal warming for milk flow is at about 40 C for about 15 minutes. Ovali, F., et al., Breastfeeding Medicine 2012, 7(6), 487. Ideally, these cold and heat therapies should be applied using the same apparatus.

Current breast thermal packs take the form of an annulus around an open center for the nipple. An opening in the form of an angular segment of a circular disk extends from the open center to the outer periphery of the pack. This opening better permits the breast pack to conform to the breast and also allows for the breast pack to be fitted around a breast pump. Representative of these circular packs is Silver U.S. Pat. No. 5,897,580. Many conventional breast packs have separate cloth or fabric covers that are uniform in composition on their front and back sides.

Improvements could be made to conventional breast packs that would permit the optimum application of both heat and cold therapy using a single device, and which would reach all of the milk glands of the breast.

### SUMMARY OF THE INVENTION

According to one aspect of the invention, a breast pack is provided for delivering heat or cold therapy to a breast. The breast pack comprises a front sheet formed as a laminate including a first fluid-impervious layer and a first fabric. The first fabric has a first loft. A back sheet is also formed as a laminate that includes a second fluid-impervious layer and a second fabric. The second fabric has a second loft that is larger than the first loft. The front sheet is bonded to the back sheet at a periphery of the breast pack, such that the front sheet and the back sheet define therebetween a volume. A gel is contained within this volume.

According to another aspect of the invention, a breast pack is provided for delivering heat or cold therapy to a breast. The breast pack comprises a front sheet formed as a laminate including a first fluid-impervious layer and a first fabric. The first fabric has a first thermal conductance. A back sheet is also formed as a laminate that includes a second fluid-impervious layer and a second fabric. The second fabric has a second thermal conductance that is smaller than the first thermal conductance. The front sheet is bonded to the back sheet at a periphery of the breast pack, such that the front sheet and the back sheet define therebetween a volume. A gel is contained within this volume.

In either of the above aspects, the first and second fluid-impervious layers may be plastic, and in particular thermoplastic films, and in embodiments are formed from a polymer selected from the group consisting of polyvinyl chloride, polypropylene, polyethylene, polycarbonate, polyurethane, polyamide and compatible mixtures thereof. One or both of the first fabric and the second fabric may be woven or nonwoven fabrics with fibers composed of polyester, nylon, spandex, cotton, viscose, flax and blends thereof. The fabrics may be mesh, knit, plush, canvas, linen, velvet or muslin. The first and second fluid-impervious layers may be directly bonded to each other at the periphery of the breast pack, or may be bonded together at the periphery using an adhesive.

In a further aspect of the invention, a method for delivering hot or cold therapy to a breast comprises providing a breast pack having a front sheet and a back sheet, the front sheet comprising a first fabric and the back sheet comprising a second fabric, with the front sheet having a first loft and a first thermal conductance and the back sheet having a second loft and a second thermal conductance. The first loft is different from the second loft, or the first thermal conductance is different from the second thermal conductance, or both. The breast pack is heated or cooled. The user selects either the front sheet or the back sheet for application to the breast. In one embodiment, the second loft is greater than the first loft. The method further includes cooling the breast pack and applying the back sheet to the breast to discourage engorgement. In another method, where the second loft is greater than the first loft, the breast pack is heated and the front sheet is applied to the breast to encourage let-down. In one embodiment, the first thermal conductance is greater than the second thermal conductance. The breast pack is cooled and the back sheet is applied to the breast to discourage engorgement. In another method, where similarly the first thermal conductance is greater than the second thermal conductance, the breast pack is heated and the front sheet is applied to the breast to encourage let-down.

The present invention provides a hot/cold breast pack where the user selects the side (back sheet or front sheet) she wants for either heat or cold therapy. She may wish to use the side with higher thermal conductance for heat therapy or cold therapy, or, alternatively, she may wish to use the side with lower thermal conductance for either heat therapy or cold therapy. The present invention affords her this choice.

The present invention permits the application of both heat and cold therapy to the human female breast using the same convenient device, with results that are superior to what has been accomplished by conventional hot/cold packs. The hot/cold breast pack is also more comfortable for the user to use.

### BRIEF DESCRIPTION OF THE DRAWINGS

Further aspects of the invention and their advantages can be discerned in the following detailed description as read in conjunction with the drawings of exemplary embodiments, in which like characters denote like parts and in which:

FIG. 1 is a perspective view of a hot/cold breast pack according to the invention, shown as applied to a human female breast;

FIG. 2 is a top view of a front sheet forming a part of the breast pack shown in FIG. 1;

FIG. 3 is bottom view of a back sheet forming a part of the breast pack shown in FIG. 1; and

FIG. 4 is a highly schematic cross sectional view of a portion of the breast pack shown in FIG. 1.

### DETAILED DESCRIPTION

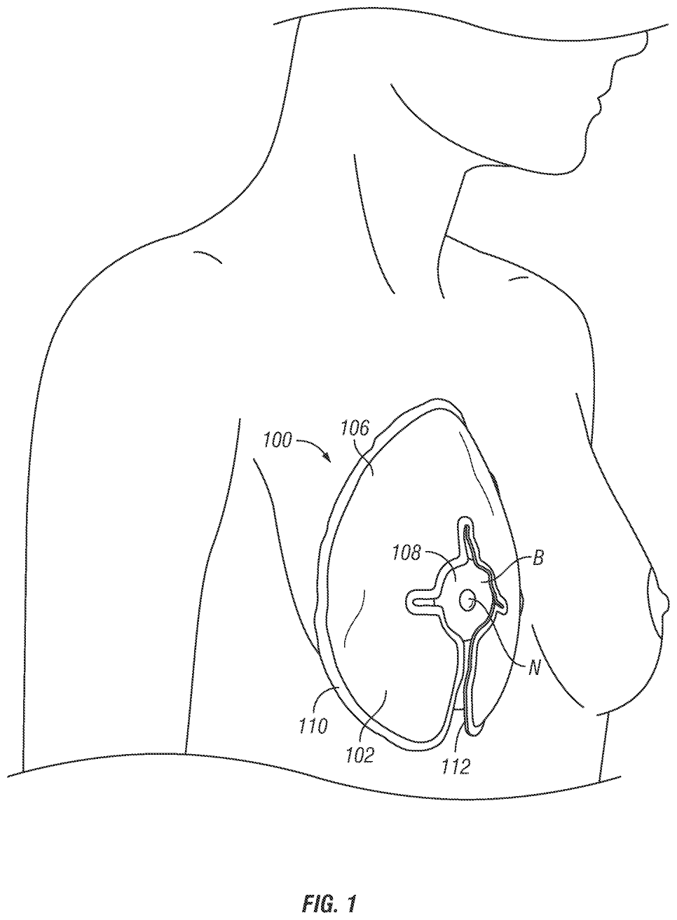

Referring to FIG. 1, a hot/cold breast pack 100 is shown as applied to a human female breast B. While, as hereinafter described, the front sheet 102 and the back sheet 104 (FIG. 3) can be flat, a body 106 of the breast pack is three dimensional and, as applied to breast B, takes on a rearwardly concave shape. Since breast B is a convex surface, this permits the body 106 to fit to breast B better. The body 106 has an open center 108 for receiving a nipple N of breast B. The open center 108 is connected to an outer periphery 110 of body 106 by an opening 112 that may take the shape of an angular segment of a circle or keyhole. The open angular segment 112 permits the body 106 to be configured roughly into a cone. The opening 112 further permits the body 106 to be fitted around a flange of a breast pump (not shown).

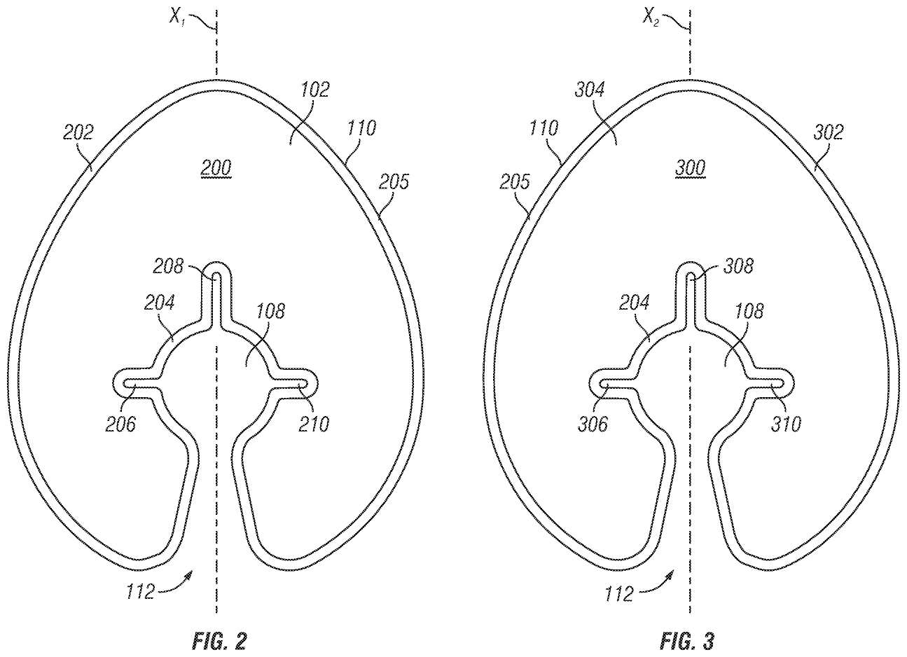

The front sheet 102 is shown in FIG. 2 while the back sheet 104 is shown in FIG. 3. In the illustrated embodiment, the front sheet 102 and the back sheet 104 have shapes that are identical to each other. Front sheet 102 is bilaterally symmetrical around a vertical axis X1 while back sheet 104 is bilaterally symmetrical around a vertical axis X2. When the front sheet 102 is bonded to the back sheet 104, a body 106 will be formed that is bilaterally symmetrical around a vertical axis. This means that the breast pack may be flipped over and, regardless of what sheet is being applied to the breast, will provide the same area coverage.

The shape of the front sheet 102 and of the back sheet 104 may take the form of a teardrop, as shown. Front and back sheets 102, 104 may be longer in a vertical direction than they are wide. In one embodiment sheets 102, 104 are about 227 mm long and are about 190 mm wide. Sheet 102 has an upper zone 200, and sheet 104 has an upper zone 300, both of which upwardly extend beyond what would otherwise be a circular margin. The teardrop shape creates better coverage of the vertical and lateral milk glands in the breast.

Front sheet 102 has a peripheral zone 202 while back sheet 104 has a peripheral zone 302. Zones 202 and 302 are the loci where the sheets 102 and 104 are bonded together. Zone 202 is continuous and not only defines the outer periphery 110 of body 106, but also an inner periphery 204 that bounds and defines open center 108. Similarly, zone 302 is continuous and defines not only the outer periphery 110 of body 106 but also the inner periphery 204 that bounds the open center 108. The outer periphery 110 and the inner periphery 204 are continuous with each other and together constitute a periphery 205 of the breast pack 100.

Open center 108 may be about 50 mm in diameter. In the illustrated embodiment, three channels 206, 208, 210; 306, 308, 310 extend radially outwardly from the open center 108 into the body 106. These channels 206, 208, 210; 306, 308, 310 permit a better conformance to breast B and aid in fitting pack 100 around a breast pump flange (not shown). The sheet peripheral zones 202, 302 surround and define these closed-end channels as well.

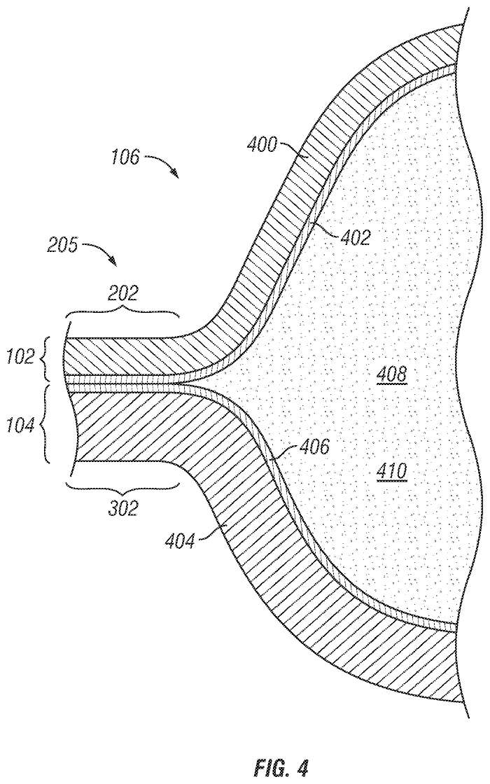

A portion of breast pack body 106 is shown in a magnified schematic sectional view in FIG. 4. The front sheet 102 is a laminate composed of at least two layers, including a first fabric layer 400 and a first fluid-impervious layer 402. The back sheet 104 is a laminate composed of at least two layers, including a second fabric layer 404 and a second fluid-impervious layer 406.

Fluid-impervious layers 402 and 406 should be impervious or fluid-tight relative to a gel 408 disposed within the volume 410 defined by front and back sheets 102, 104. Fluid-impervious layers 402 and 406 may be plastic, and more particularly may be a thermoplastic film. The thermoplastic film may be made of a polymer selected from the group consisting of polyvinyl chloride, polypropylene, polyethylene, polycarbonate, polyurethane, polyamide and compatible mixtures, compounds and derivatives thereof. In the illustrated embodiment, layers 402 and 406 are directly and hermetically bonded together throughout zones 202 and 302, sealing together sheets 102 and 104. In an alternative embodiment, the layers 402 and 406 may be sealed together using an adhesive (not shown).

Layer 402 is laminated, bonded or fused with first fabric layer 400, while layer 406 is laminated, bonded or fused with second fabric layer 404. The ends of fibers making up fabric layer 400 may extend into fluid barrier layer 402, while the ends of fibers making up fabric layer 404 may extend into fluid barrier layer 406.

In the illustrated embodiment, the loft or thickness of fabric layer 400 is less than the loft or thickness of fabric layer 404. Layer 400 may be knitted fabric, while layer 404 may be plush fabric. Importantly, the thermal conductance of layer 400 is greater than the thermal conductance of layer 404. This may be achieved in various ways. First, and as illustrated, the loft of layer 400 may be significantly less than the loft of layer 404. This will mean that layer 404 will have more air spaces and will act more as a heat insulator than layer 404, even if the fabrics are made of the same material.

Second, the composition of the fibers making up fabric layers 400 and 404 may be chosen to be different from each other, and so as to have different thermal conductivities. The thermal conductivity of nylon is generally in the range of 0.25 to 0.5 W/m K. That of polyester is typically in the range of 0.15 to 0.35 W/m K. The thermal conductivity of cotton is generally in the range of 0.05 to 0.07 W/m K. Fabric layer 400 may be chosen to be composed of a fiber (or more than one of them) having a higher thermal conductivity than that of a fiber (or more than one of them) used to make up fabric layer 404. Having a higher thermal conductivity will mean that the layer will have a higher conductance, and this will be the case even if the weave or loft of the layers 400, 404 are otherwise the same. It is also possible to combine the effects in difference in loft and thermal conductivity to achieve the desired difference in thermal conductance between layers 400 and 404.

In the illustrated embodiment, polyester is used to make up both relatively smooth layer 400 and relatively plush or thick fabric layer 404. Layers 400, 404 alternatively could be constructed of any woven or nonwoven fabric, using fibers composed of polyester, cotton, nylon, spandex, viscose, flax and blends thereof. The fabrics may be knit, plush, canvas, mesh, linen, velvet or muslin.

Conventional breast hot/cold packs use separate smooth nylon covers which, when used for cold therapy, feel extremely cold and uncomfortable to the skin. The inventors have found that by decreasing the thermal conductance of the sheet used for cold therapy, a more comfortable experience is obtained.

The gel 408 may be composed of water, up to 10 weight percent of a cellulose type thickener such as sodium methylcellulose (CMC), up to 30 weight percent of a lower molecular weight glycol and up to 2 weight precent of a preservative or biocide suitable for a water-based composition. The low molecular weight glycol may be propylene glycol. The preservative may be methylchlorothiazolinone or methylisothiazolinone. The total weight of the gel should be at least 200 g and more preferably is at least 220 g. As so filled, the body 106 is nevertheless discreet and not bulky.

Hot/cold packs according to the invention were prepared and tested for cooling and warming performance. The fabrics used were knitted and plush polyester fabric, both as fused with a PVC layer internal to the fabric layer. The amount of gel fill was selected as either 200 or 220 g.

For warming performance, the hot/cold packs were heated in a microwave at 700 W for 50 sec. to a temperature of 50 C. For cooling performance, the hot/cold packs were cooled in a freezer for up to six hours to a temperature of −10 C. The hot/cold packs were then placed on a hot plate heated at a constant temperature of 37 C, to emulate a human breast. A temperature probe was placed between the test side (either knitted or plush) and the heated plate. A control thermocouple measured the temperature of the hot plate to ensure that it was constant. Temperature readings were collected every 3 seconds, for at least 1.5 hours. The results are tabulated below. For cooling performance, the total cooling time was reported from when the temperature decrease stabilized (after about 10 readings) to the time the temperature reached 18 C. For warming performance, the total warming time was reported from when the temperature increase stabilized (after about 10 readings) to the time the temperature reached about 39 C.

### Cooling Performance

| | | Range of | Least duration |
|---|---|---|---|
| Weight (g) | Testing side | temperature, ° C. | (min) |
| 200 | Knitted fabric | 6.8-18.0 | 13 |
| 200 | Plush fabric | 0.10-18.04 | 16 |
| 220 | Knitted fabric | 5.3-18.0 | 16 |
| 220 | Plush fabric | 0.94-18.05 | 20 |

### Warming (Thermal) Performance

| | | Range of | Least duration |
|---|---|---|---|
| Weight (g) | Testing side | temperature, ° C. | (min) |
| 200 | Knitted fabric | 49.97-39.36 | 30 |
| 200 | Plush fabric | 47.87-39.26 | 30 |
| 220 | Knitted fabric | 49.36-39.36 | 30 |
| 220 | Plush fabric | 48.25-39.31 | 30 |

If optimal cooling for mitigating engorgement is 10-18° C. for 15-20 minutes and if optimal warming for milk flow is 40° C. for 15 minutes, then the above data demonstrate that the combined hot/cold breast pack according to the invention is therapeutically effective for both cooling and warming treatments. The plush fabric side or sheet of the breast pack cools the breast while remaining at a temperature within the range of about 0 to about 18° C. for at least 16 minutes, and in the 220 g embodiment, the temperature is held to this range for at least 20 minutes. The knitted fabric side or sheet of the breast pack warms the breast while remaining at a temperature within the range of about 50 to about 39° C. for at least 30 minutes.

In summary, a hot and cold breast pack has been shown and described in which the thermal conductance of the front and back sheets of the pack are selected to be intentionally different from each other. This promotes comfort and versatility in use.

While an illustrated embodiment of the present invention have been described and illustrated in the appended drawings, the present invention is not limited thereto but only by the scope and spirit of the appended claims.

We claim:

1. A breast pack for delivering heat or cold therapy to a breast, the breast pack comprising:
a front sheet formed as a laminate including a first fluid-impervious layer and a first fabric, the first fabric having a first loft;
a back sheet formed as a laminate including a second fluid-impervious layer and a second fabric, the second fabric having a second loft larger than the first loft;
the front sheet bonded to the back sheet at a periphery of the breast pack, such that the front sheet and the back sheet define therebetween a volume, a gel contained within the volume.

2. The breast pack of claim 1, wherein the first fluid-impervious layer is plastic.

3. The breast pack of claim 2, wherein the plastic is a thermoplastic film.

4. The breast pack of claim 3, wherein the thermoplastic film is selected from the group consisting of polyvinyl chloride, polypropylene, polyethylene, polycarbonate, polyurethane, polyamide and compatible mixtures thereof.

5. The breast pack of claim 1, wherein the second fluid-impervious layer is plastic.

6. The breast pack of claim 5, wherein the second fluid-impervious layer is a thermoplastic film.

7. The breast pack of claim 6, wherein the thermoplastic film is selected from the group consisting of polyvinyl chloride, polypropylene, polyethylene, polycarbonate, polyurethane, polyamide and compatible mixtures thereof.

8. The breast pack of claim 1, wherein the first fabric is a woven or nonwoven fabric composed of fibers selected from the group consisting of polyester, nylon, spandex, cotton, viscose, flax and blends thereof.

9. The breast pack of claim 1, wherein the first fabric is mesh, knit, plush, canvas, linen, velvet or muslin.

10. The breast pack of claim 1, wherein the second fabric is a woven or nonwoven fabric composed of fibers selected from the group consisting of polyester, nylon, spandex, cotton, viscose, flax and blends thereof.

11. The breast pack of claim 1, wherein the second fabric is mesh, knit, plush, canvas, linen, velvet or muslin.

12. The breast pack of claim 1, wherein the first fluid-impervious layer is directly bonded to the second fluid-impervious layer at the periphery.

13. The breast pack of claim 1, wherein the front sheet has a first shape, the back sheet has a second shape, and the first shape is the same as the second shape.

14. The breast pack of claim 13, wherein the first and second shapes are a teardrop shape.

15. The breast pack of claim 13, wherein the first shape and the second shape are bilaterally symmetrical around a vertical axis.

16. The breast pack of claim 13, wherein the first and second shapes are formed around an open center, the periphery of the breast pack including an outer periphery, an opening in the form of an angular segment extending from the center to the outer periphery, such that the breast pack may better fit to the breast.

17. A breast pack for delivering heat or cold therapy to a breast, the breast pack comprising:
a front sheet formed as a laminate including a first fluid-impervious layer and a first fabric, the first fabric having a first thermal conductance;
a back sheet formed as a laminate including a second fluid-impervious layer and a second fabric, the second fabric having a second thermal conductance smaller than the first thermal conductance;
the front sheet bonded to the back sheet, at a periphery of the breast pack, such that the front sheet and the back sheet define a volume therebetween, a gel contained within the volume.

18. The breast pack of claim 17, wherein the first fluid-impervious layer is plastic.

19. The breast pack of claim 18, wherein the first fluid-impervious layer is a thermoplastic film.

20. The breast pack of claim 19, wherein the thermoplastic film is made of a polymer selected from the group consisting of polyvinyl chloride, polypropylene, polyethylene, polycarbonate, polyurethane, polyamide and compatible mixtures thereof.

21. The breast pack of claim 17, wherein the second fluid-impervious layer is plastic.

22. The breast pack of claim 21, wherein the second fluid-impervious layer is a thermoplastic film.

23. The breast pack of claim 22, wherein the thermoplastic film is formed from a polymer selected from the group consisting of polyvinyl chloride, polypropylene, polyethylene, polycarbonate, polyurethane, polyamide and compatible mixtures thereof.

24. The breast pack of claim 17, wherein the first fluid-impervious layer is directly bonded to the second fluid-impervious layer at the periphery.

25. The breast pack of claim 17, wherein the first fabric is a woven or nonwoven fabric composed of fibers selected from the group consisting of polyester, nylon, cotton, spandex, viscose, flax and blends thereof.

26. The breast pack of claim 17, wherein the first fabric is mesh, knit, plush, canvas, linen, velvet or muslin.

27. The breast pack of claim 17, wherein the second fabric is a woven or nonwoven fabric composed of fibers selected from the group consisting of polyester, nylon, cotton, spandex, viscose, flax and blends thereof.

28. The breast pack of claim 17, wherein the second fabric is plush, knit, mesh, canvas, linen, velvet or muslin.

29. A method for delivering hot or cold therapy to a breast, the method comprising the steps of:
providing a breast pack having a front sheet and a back sheet, the front sheet comprising a first fabric and the back sheet comprising a second fabric, the first fabric having a first loft and a first thermal conductance and the second fabric having a second loft and a second thermal conductance, wherein the first loft is different from the second loft, the first thermal conductance is different from the second different from the second thermal conductance, or both;
heating or cooling the breast pack;
selecting one of the front sheet and the back sheet for application to the breast; and
applying the selected one of the front sheet and the back sheet to the breast.

30. The method of claim 29, wherein the second loft is greater than the first loft, the method including the further steps of:
cooling the breast pack; and
applying the back sheet to the breast to discourage engorgement.

31. The method of claim 29, wherein the second loft is greater than the first loft, the method including the further steps of:
heating the breast pack; and
applying the front sheet to the breast to encourage let-down.

32. The method of claim 29, wherein the first thermal conductance is greater than the second thermal conductance, the method including the further steps of:
heating the breast pack; and
applying the front sheet of the breast pack to the breast to encourage let-down.

33. The method of claim 29, wherein the first thermal conductance is greater than the second thermal conductance, the method including the further steps of:
cooling the breast pack; and
applying the back sheet of the breast pack to the breast to discourage engorgement.
